(19) United States
(12) Patent Application Publication
(10) Pub. No.: US 2025/0213395 A1
(43) Pub. Date: Jul. 3, 2025

(54) LIGHT-SHIELDING EYE MASK

(71) Applicant: SheungHo LI, Hong Kong (CN)

(72) Inventor: SheungHo LI, Hong Kong (CN)

(21) Appl. No.: 18/982,414

(22) Filed: Mar. 22, 2025

(51) Int. Cl. A61F 9/04

(52) U.S. Cl. CPC A61F 9/045

(57) ABSTRACT

The present disclosure relates to the technical field of eye masks, and in particular, to an improved light-shielding eye mask, including a surface protective layer, an eye contact layer, and a fixing strap. Left and right sides of the surface protective layer are respectively connected to the fixing strap. The fixing strap is provided with an adjustment buckle for adjusting the length of the fixing strap, making it easy to adjust the tightness of the eye mask. The side edges of the periocular contact surface of the eye mask are designed in a vertical I-shape with a relatively thin thickness, bringing less sense of foreign body when sleeping on the side and better comfort.

TECHNICAL FIELD

The present disclosure relates to the technical field of eye masks, and in particular, to an improved light-shielding eye mask.

### BACKGROUND

Due to various reasons, modern people have irregular lives, lack of sleep at night and lack of light-free sleeping environment during the day, resulting in listlessness during the day and low work efficiency. In the long run, it is easy to cause disorganization of the biological clock and endocrine dyscrasia, which seriously affects the health of bodies. Eye masks can provide a light-free sleeping environment for daytime sleepers.

Eye masks, which refer to things worn on the eyes to play a protective or shielding role, have become fashionable supplies, and play an important role in eliminating fatigue and assisting sleep in the life of travel. It is very important to choose a right eye mask. A fixing strap of the eye mask that is too loose can easily slip during sleep, causing the eyes to be irritated by light. A fixing strap that is too tight can easily compress the nerves around the eyeballs, affecting blood circulation. In addition, the comfort of using the eye mask is also very important. Wearing the eye mask uncomfortably will also affect the quality of sleep.

Nowadays, most of the fixing straps of eye masks are adjusted and fixed by means of Velcro, but the Velcro is not tightly fixed enough and easy to loosen, with relatively short durability and service life. Secondly, the existing eye masks are in direct contact with the entire eyes, which will cause a sense of pressure on the entire eyes. Some eye masks are only in contact with the eye periphery, with the side parts near the temple designed relatively thick, so that there will be a sense of foreign body when wearing the eye masks and sleeping on the side, and discomfort may also occur on the side of the eye periphery.

### SUMMARY

In order to solve the above problems, the present disclosure provides an improved light-shielding eye mask, which can adjust the appropriate tightness according to the situation, has good air permeability, and is comfortable to wear.

In order to achieve the above objects, the technical solution adopted by the present disclosure is: An improved light-shielding eye mask, including a surface protective layer, an eye contact layer, and a fixing strap, where left and right sides of the surface protective layer are respectively connected to the fixing strap, the fixing strap is provided with an adjustment buckle for adjusting the length of the fixing strap; one side of the eye contact layer is flat, the other side is concave, and the side edge is a convex periocular contact surface; the flat side of the eye contact layer is provided on the back of the surface protective layer; the periocular contact surface includes a left contact portion and a right contact portion that are symmetrically arranged, and both the left end side edge of the left contact portion and the right end side edge of the right contact portion are arranged in a vertical I-shape.

Further, the eye contact layer is detachably fixed to the surface protective layer by means of Velcro, and the surface of the eye contact layer is made of Lycra fabric with the interior filled with highly elastic sponge.

Further, the left contact portion and the right contact portion are designed as independent individuals, and are detachably fixed to the surface protective layer by means of Velcro, respectively.

Both the right end side edge of the left contact portion and the left end side edge of the right contact portion are arc-shaped.

Further, the left contact portion and the right contact portion may also be integrally designed, the right side of the left contact portion and the left side of the right contact portion are integrally connected, and the recessed parts are connected.

Further, the fixing strap is an elastic fixing strap, both ends of the surface protective layer are sewn with connecting straps which are provided with connecting rings, one end of the fixing strap is fixed to the connecting ring at one end of the surface protective layer, and the other end of the fixing strap is fixed to the adjustment buckle after passing through the adjustment buckle and the connecting ring at the other end of the surface protective layer in turn.

The side edges of the surface protective layer are hemmed and seamed, and the lower side of the surface protective layer is provided with a concave arc that avoids the dorsum of the nose.

The beneficial effects of the present disclosure lie in that: According to the improved light-shielding eye mask of the present disclosure, an adjustment buckle is used to adjust the tightness of the fixing strap, bringing good stability and longer service life; the side edges of the periocular contact surface of the eye mask are designed in a vertical I-shape with a relatively thin thickness, bringing less sense of foreign body when sleeping on the side and better comfort; in addition, the eye contact layer is filled with highly elastic sponge, and the surface is made of Lycra fabric, bringing good elasticity, good touch when wearing, and good comfort.

### BRIEF DESCRIPTION OF THE DRAWINGS

FIG. 1 is a schematic diagram of the back of the overall structure of this embodiment.

FIG. 2 is a schematic diagram of the front of the overall structure of this embodiment.

FIG. 3 is a schematic diagram of the back of a surface protective layer and an eye contact layer in this embodiment.

FIG. 4 is a schematic diagram of the front of the surface protective layer and the eye contact layer in this embodiment.

FIG. 5 is a schematic diagram of the back of the surface protective layer and the fixing strap in this embodiment.

FIG. 6 is a schematic diagram of the integrated design of a left contact portion and a right contact portion in this embodiment.

Explanation of reference numerals: 1. surface protective layer; 11. connecting strap; 12. connecting ring; 2. eye contact layer; 21. periocular contact surface; 211. I-shape; 22. left contact portion; 23. right contact portion; 3. fixing strap; 4. adjustment buckle; 5. Velcro.

### DETAILED DESCRIPTION

The present disclosure will be further described in detail below through specific implementations in conjunction with the accompanying drawings. This application can be implemented in many different forms and is not limited to the implementation described in this embodiment. The purpose of providing the following specific implementations is to facilitate a clearer and more thorough understanding of the disclosed content of this application. The words indicating directions such as up, down, left, and right are merely for the positions of the structures shown in the corresponding accompanying drawings.

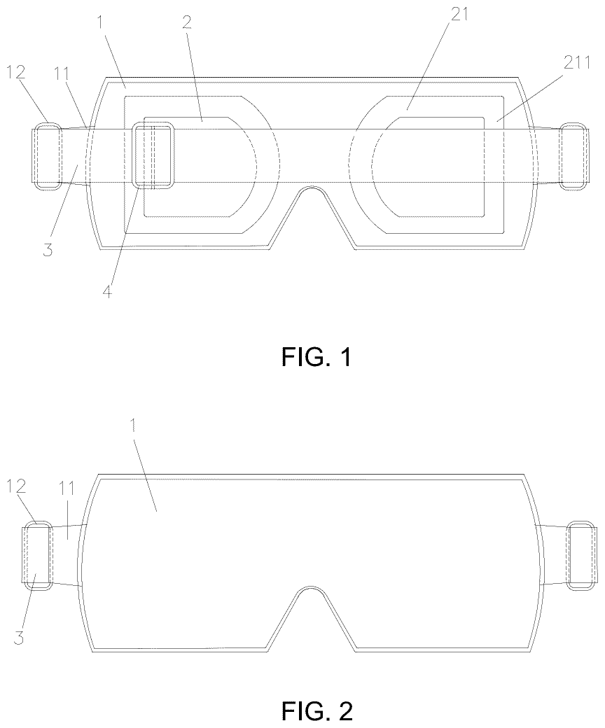

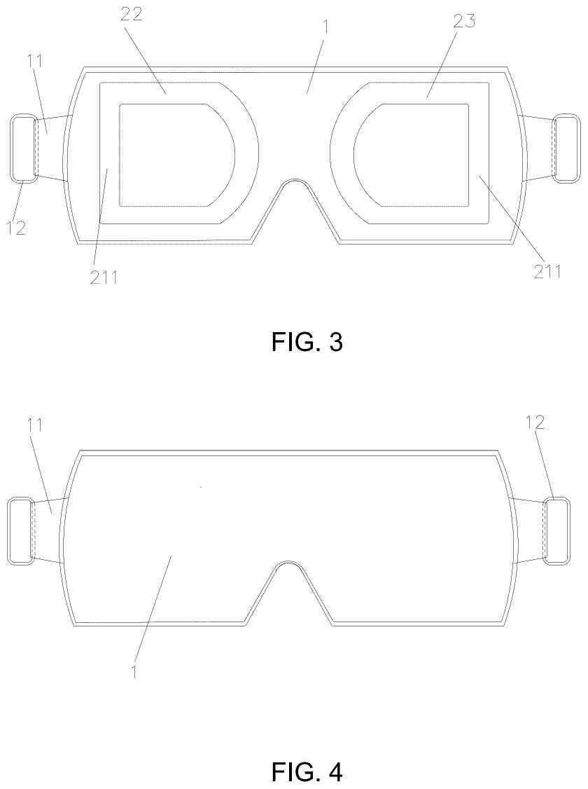

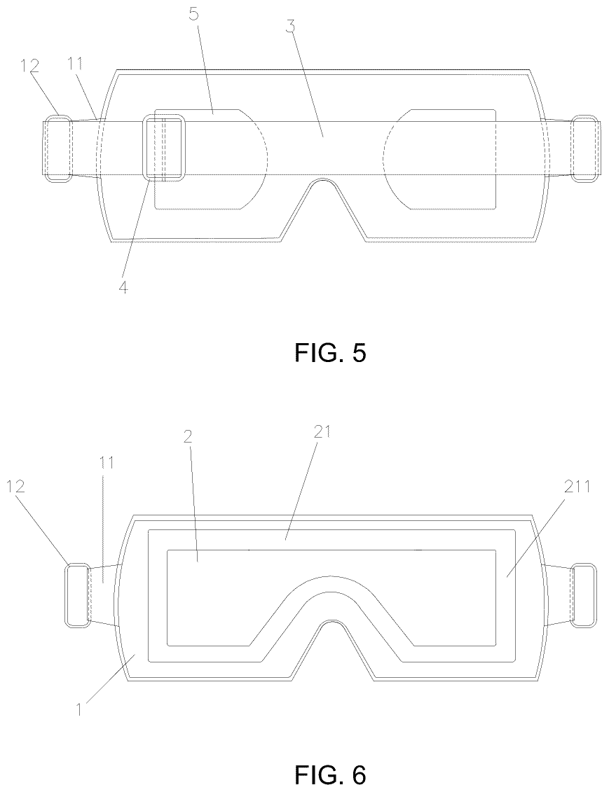

Referring to FIGS. 1-5, the present disclosure relates to an improved light-shielding eye mask, including a surface protective layer 1, an eye contact layer 2, and a fixing strap 3. Left and right sides of the surface protective layer 1 are respectively connected to the fixing strap 3, and the fixing strap 3 is provided with an adjustment buckle 4 for adjusting the length of the fixing strap 3. One side of the eye contact layer 2 is flat, the other side is concave, and the side edge is a convex periocular contact surface 21. The flat side of the eye contact layer 2 is provided on the back of the surface protective layer 1. The periocular contact surface 2 includes a left contact portion 22 and a right contact portion 23 that are symmetrically arranged, and both the left end side edge of the left contact portion 22 and the right end side edge of the right contact portion 23 are arranged in a vertical I-shape 211.

In this embodiment, the eye contact layer 2 is detachably fixed to the surface protective layer 1 by means of Velcro 5, facilitating easy removal for cleaning or replacement, and at the same time, the position of the eye contact layer 2 can be adjusted according to the needs of different users. The surface of the eye contact layer 2 is made of Lycra fabric with the interior filled with highly elastic sponge. The Lycra fabric has good elasticity and feels good when wearing, and the filling of highly elastic sponge makes the periocular contact surface 2 have slight elastic contact with the eye area, with no pressure and good comfort.

The left contact portion 22 and the right contact portion 23 are designed as independent individuals. Both the right end side edge of the left contact portion 22 and the left end side edge of the right contact portion 23 are arc-shaped. The left contact portion 22 and the right contact portion 23 are detachably fixed to the surface protective layer 1 by means of Velcro 5, respectively, and the positions of the left contact portion 22 and the right contact portion 23 can be adjusted according to the distance between the eyes of different users.

In addition, referring to FIG. 6, the left contact portion 22 and the right contact portion 23 may also be integrally designed. The right side of the left contact portion 22 and the left side of the right contact portion 23 are integrally connected, and the recessed parts are connected.

In this embodiment, the fixing strap 3 is an elastic fixing strap 3. Both ends of the surface protective layer 1 are sewn with connecting straps 11 which are provided with connecting rings 12. One end of the fixing strap 3 is fixed to the connecting ring 12 at one end of the surface protective layer 1, and the other end of the fixing strap 3 is fixed to the adjustment buckle 4 after passing through the adjustment buckle 4 and the connecting ring 12 at the other end of the surface protective layer 1 in turn. The connecting straps 11 are provided at both ends of the surface protective layer 1 and are connected to the fixing strap 3 through connecting rings 12. In order not to directly use the connecting rings 12 at turning points between the surface protective layer 1 and the fixing strap 3, the connecting straps 11 are added for buffering so that the connecting straps 11 are located at the temple with no sense of foreign body, making it more comfortable to wear. The adjustment buckle 4 can adjust the length of the fixing strap 3 to be used by different users. In addition, the fixing strap 3 is made of elastic material, making it more stable to wear.

The side edges of the surface protective layer 1 are hemmed and seamed to make the periphery of the eyes more tidy and durable. The lower side of the surface protective layer 1 is provided with a concave arc that avoids the dorsum of the nose. This design can make it more comfortable for users to wear and has a better fit.

Compared to the prior art, according to the improved light-shielding eye mask of the present disclosure, the adjustment buckle 4 is used to adjust the tightness of the fixing strap 3, bringing good stability and longer service life; the side edges of the periocular contact surface 21 of the eye mask are designed in a vertical I-shape 211 with a relatively thin thickness of 0.5 cm-1.4 cm, bringing less sense of foreign body when sleeping on the side and better comfort; in addition, the eye contact layer 2 is filled with highly elastic sponge, and the surface is made of Lycra fabric, bringing good elasticity, good touch when wearing, and good comfort.

It should be further noted that, unless otherwise expressly stipulated and limited, “provided”, “fixed” and other terms should be understood in a broad sense. For those of ordinary skill in the art, the specific meaning of the above terms in the present disclosure can be understood according to specific circumstances.

The above implementations are merely descriptions of the preferred implementations of the present disclosure and do not limit the scope of the present disclosure. Without departing from the design spirit of the present disclosure, various deformations and improvements made to the technical solution of the present disclosure by ordinary engineers and technicians in the field shall fall within the scope of protection determined by the claims of the present disclosure.

1. An improved light-shielding eye mask, comprising a surface protective layer, an eye contact layer, and a fixing strap, wherein left and right sides of the surface protective layer are respectively connected to the fixing strap, the fixing strap is provided with an adjustment buckle for adjusting the length of the fixing strap; one side of the eye contact layer is flat, the other side is concave, and the side edge is a convex periocular contact surface; the flat side of the eye contact layer is provided on the back of the surface protective layer; the periocular contact surface comprises a left contact portion and a right contact portion that are symmetrically arranged, and both the left end side edge of the left contact portion and the right end side edge of the right contact portion are arranged in a vertical I-shape.

2. The improved light-shielding eye mask according to claim 1, wherein the eye contact layer is detachably fixed to the surface protective layer by means of Velcro, and the surface of the eye contact layer is made of Lycra fabric with the interior filled with highly elastic sponge.

3. The improved light-shielding eye mask according to claim 2, wherein the left contact portion and the right contact portion are designed as independent individuals, and are detachably fixed to the surface protective layer by means of Velcro, respectively.

4. The improved light-shielding eye mask according to claim 3, wherein both the right end side edge of the left contact portion and the left end side edge of the right contact portion are arc-shaped.

5. The improved light-shielding eye mask according to claim 2, wherein the left contact portion and the right contact portion are integrally designed, the right side of the left contact portion and the left side of the right contact portion are integrally connected, and the recessed parts are connected.

6. The improved light-shielding eye mask according to claim 1, wherein the fixing strap is an elastic fixing strap, both ends of the surface protective layer are sewn with connecting straps which are provided with connecting rings, one end of the fixing strap is fixed to the connecting ring at one end of the surface protective layer, and the other end of the fixing strap is fixed to the adjustment buckle after passing through the adjustment buckle and the connecting ring at the other end of the surface protective layer in turn.

7. The improved light-shielding eye mask according to claim 1, wherein the side edges of the surface protective layer are hemmed and seamed, and the lower side of the surface protective layer is provided with a concave arc that avoids the dorsum of the nose.
